(19) United States
(12) Patent Application Publication
(10) Pub. No.: US 2007/0119270 A1
(43) Pub. Date: May 31, 2007

(54) Rapid soil drying

(76) Inventors: Gordon Shanthakumar Rajendram, Hamilton (NZ); Dale Frew, Mount Cook (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/574,127

(22) PCT Filed: Oct. 1, 2004
(86) PCT No.: PCT/NZ04/00238

§ 371 Date: Mar. 31, 2006

(30) Foreign Application Priority Data
Oct. 3, 2003 (NZ) 528638

(51) Int. Cl. G01N 1/44; G01N 33/24

(52) U.S. Cl. 73/863.12

(57) ABSTRACT

A method and assembly are described to dry soil for use in testing a variety of physical and chemical parameters such as phosphorus content. The method broadly involves the steps of particle size reduction, subjecting the sample to an airflow and also heating of the soil sample. It is the inventors experience that by use of the method of the present invention, samples can be prepared for accurate analysis with as little as 20 minutes rather than the more traditional methods which take 20 to 24 hours.

### TECHNICAL FIELD

This invention is related to rapid soil drying. The present invention discloses a method and device that can be used prior to the measurement of chemical and physical properties within soil.

### BACKGROUND ART

Soil testing is a common occurrence for a variety of industries including farming. In farming, it is desirable to know the levels of various soil constituents such as potassium, magnesium, sodium, calcium, phosphorus and sulphur so that, for example fertiliser is applied at correct concentration and frequency. Other testing applications include soil testing of constructions sites, industrial sites such as chemical processing facilities and mining sites, for example to determine if contamination has occurred from chemicals or heavy metals.

Current testing practice for determining key nutrient levels in soil is carried out in laboratories where samples are prepared for analysis. It is standard to firstly prepare the soil sample via forced drying or moisture removal. By removing moisture from the soil sample, the sample becomes stabilised physically and chemically and key properties are less likely to alter over time. Changes that may occur if drying does not occur include mineralisation of some nutrients and soil pH variation. Traditionally, samples are dried to a point where there is minimal residual moisture—i.e. if the sample was re-dried, there would be a negligible difference in weight before and after re-drying.

At present, soil cores are collected in the field and then transported to laboratories where they are kept intact and dried overnight (for at least 20 hours) at temperatures of 30 to 35° C. After oven drying, (usually on the next day), the samples are ground and passed through a 2 mm sieve, at which point the samples are then ready for chemical or physical analysis.

Alternative methods of drying such as freeze drying and microwave drying are not generally used in standard laboratory testing. Both of these alternative methods are comparatively expensive and require specialised equipment and operation.

The soil samples, termed ‘cores’ used in soil analysis are intact plugs of soil approximately 2.5×7.5 cm in size (for agricultural testing) and 2.5×15 cm in size (for horticultural testing). The cores are used primarily to determine the nutrient status of the soil. A standard recommendation is that 15 to 20 cores are taken from the area where the nutrient status of the soil is to be measured. By taking a number of samples, local variations can be averaged or omitted.

A key drawback of the above standard soil preparation practice is that at least one day is lost before chemical analysis can commence. This is a problem as decisions regarding for example pasture fertilisation are delayed.

A further disadvantage of present practice is that samples must be transported to a remote site i.e. the laboratory. Besides the extra cost of transport, this additional step introduces possible contamination of the samples e.g. through mishandling or exposure of the samples to heat or moisture during transportation.

Despite the above disadvantages of prior methods, alternative methods of testing are seldom considered because:
Analysis methods are very standardised and there is some tradition associated with particular methods;
Existing methods have been established through a great deal of investment in time and money and enshrined in various quality standards hence a low motivation to experiment with other methods;
The degree of accuracy expected from a laboratory test is higher than that required for many practical applications such as making a decision regarding fertiliser application.

To address the disadvantages of existing methods, it is therefore highly advantageous if soils can be dried rapidly, without compromising chemical and physical test results so that test results can be obtained more quickly.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF INVENTION

According to one aspect of the present invention there is provided a method for drying soil including the steps of:
(a) increasing the surface area of the soil;
(b) forcing a substantially inert gas through the soil;
(c) subjecting the soil to an elevated temperature.

In preferred embodiments, the samples are in order for analysis after approximately 1 hour. More preferably, the samples are ready for analysis after approximately 20 minutes.

Most preferably, the moisture content after completion of steps (a) to (c) is less than approximately 9% wt.

The present invention relates to a method of drying soil in a manner that removes moisture from the soil, whilst also substantially not altering chemical and/or physical characteristics of the soil, other than removal of moisture (water).

In one embodiment, steps (a) to (c) as described above may be performed sequentially. In alternate embodiments, steps (a) and (b), (b) and (c), (a) and (c), or (a), (b) and (c) may be performed at substantially the same time.

In preferred embodiments, the speed of drying may be substantially more rapid when compared to prior art methods (which take 20 to 24 hours to dry). It has been found by the inventor that the speed of drying may be reduced to less than approximately one hour. More preferably, the speed for drying may be less than approximately 20 minutes. Those skilled in the art should appreciate that the rate of drying may be dependent on the soil type. It is the inventor's experience that clay soils tend to take the longest to dry whereas sandy soils are by comparison, quicker to dry.

The present invention may be used for soils taken from a wide variety of sites. In preferred embodiments, the soil may be a sample taken from arable land. In other embodiments, soil may be taken from construction sites, forestry sites, or industrial manufacturing facilities. However this list should not be seen as limiting as it should be appreciated by those skilled in the art that other sites may be analysed with out departing from the scope of the invention.

It should be appreciated by those skilled in the art that the drying method may be performed at the test site (in-situ) or in a laboratory or other testing facility. In-situ testing has the advantage that potential errors due to the mishandling or sample exposure are minimised. Also transport costs are eliminated by samples being able to be tested on-site rather than having to be transported to a laboratory.

It is the inventors understanding that the present invention is robust enough that it may be used for all varieties of soil types. The fact that the present invention removes moisture rapidly from the sample without altering the measured chemical and/or physical characteristics of the soil is a critical factor in laboratory analysis where the sample, when measured, must still be representative of the area from which the sample was taken. Characteristics of particular importance for measurement may include the level of phosphorus (or Olsen P), sulphur, heavy metals, potassium, magnesium, sodium and calcium and other elements or compounds that are routinely required to be analysed. Further characteristics include: the degree of elasticity of the soil sample or friability/texture properties of the soil generally.

Preferably, the increase in surface area may be achieved by breaking the soil down (‘breakdown’) into smaller particles by mechanical motion, for example by hand; or in a machine, for example, by pressing the soil through a sieve. Most preferably, the mean particle size after breakdown may be substantially less than 10 mm. It should be appreciated that the soil need not be of a uniform particle size. It is the inventor's experience that a reduced particle size increases the speed with which moisture is removed from the soil particles.

In preferred embodiments, the inert gas may be air. Most preferably, the gas may be moisture free. In alternative embodiments, the method may include gas conditioners such as a dehumidifier step and/or use of a desiccating gel to remove moisture from the gas prior or during use in the present invention. Those skilled in the art should appreciate that the use of dry air mimics the effect of wind drying.

Preferably, gas may be forced across the soil particles. In general, the air is fan forced. Most preferably the gas flow may be less than 4 m/s. Most preferably, the flow may be approximately 2 m/s.

Preferably, the temperature to which the soil may be elevated is high enough to allow sample drying without impacting on the chemical and/or physical properties to be measured. In the inventor's experience this temperature may be critical and preferably, the temperature range varies from approximately 20° C. to 50° C., although lower temperatures are also envisaged. It is likely that temperatures above approximately 50° C. result in not only moisture loss, but also deterioration of the chemical and/or physical structure of the soil. In preferred embodiments, the temperature to which the soil may be elevated varies from approximately 30° C. to 40° C. Most preferably the temperature may be approximately 35° C.

In a further embodiment, the drying equipment may be preheated before step (c).

In an alternative embodiment, the method may also include a further step (d) of:
(d) moving the soil.

Step (d) occurs at the same time as steps (a) to (c) or may only occur during steps (b) or (c) or both steps (b) and (c).

Most preferably, the particles remain in motion for substantially all of the drying time. In an alternative embodiment, particles may only be kept in motion for a discrete portion of time and/or discrete portions of time.

Methods envisaged by the inventor for keeping the soil in motion may include tossing, vibration, oscillation or shaking the soil in a dish or in a container or containers such as a container or series of containers, either in series or nested within each other.

According to a further aspect of the present invention there is provided an assembly for drying of soil which includes:
(a) an inert gas supply device which is capable of forcing inert gas through a soil sample;
(b) a heating element which is capable of subjecting the soil to an elevated temperature.

Preferably, the assembly described above further includes a soil crusher device which is capable of increasing the surface area of the soil.

Preferably, the assembly described above further includes a device capable of keeping the soil in motion.

From the above description, those skilled in the art should appreciate that the invention offers a fast alternative to present soil drying methods, allowing for faster testing of soil samples. The method includes the steps of increasing particle surface area, forced air circulation and elevated temperature. A device is also described which incorporates the above steps. As the process is quick and the device simple, measurements can be made in-situ to avoid complications of transporting the sample to a laboratory whilst still obtaining accurate results.

### BRIEF DESCRIPTION OF DRAWINGS

Further aspects of the present invention will become apparent from the following description which is given by way of example only and with reference to the accompanying drawings in which:

FIG. 1 is a drawing of a soil core sample;

FIG. 2 is a drawing of soil core samples on a sieve; and,

FIG. 3 is a drawing of a sub-sample from the core samples.

### BEST MODES FOR CARRYING OUT THE INVENTION

Non-limiting examples illustrating the invention will now be provided. It will be appreciated that the below description is provided by way of example only and variations in materials and technique used which are known to those skilled in the art are contemplated.

In order to determine if there may be a difference in key nutrient results, tests were completed where soils were dried at differing rates. Soil core samples are currently oven dried at 30-35° C. overnight (for 20-24 hours) and control samples using this method of drying were used for comparison.

Soils encompassing many soil groups were collected for analysis. These soils were sieved and mixed thoroughly.

### EXAMPLE 1

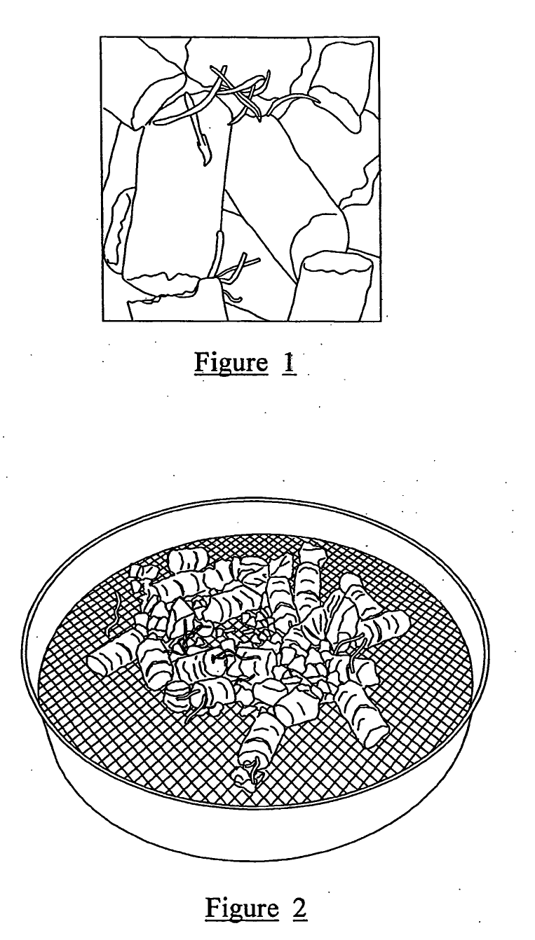

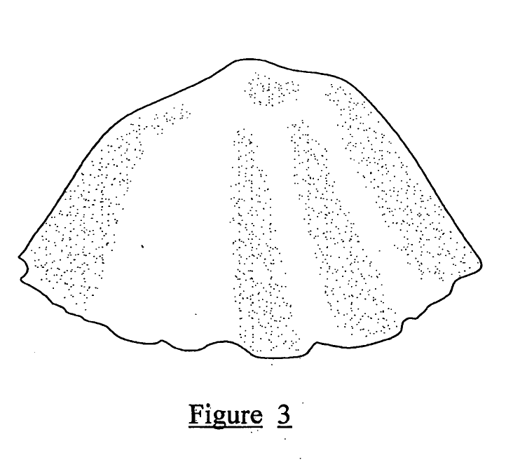

Referring to FIG. 1, core samples 1 of granular soil (clay loam) were received (soil samples 1A and 1B as shown in the table below) and placed into a 2 mm sieve 2 as shown in FIG. 2. The soil core samples were broken down and forced through the sieve to reduce the particle surface area. A sub-sample 3 (labelled 1B) was then taken as shown in FIG. 3 which was then placed into a soil dryer of the present invention (not shown) and dried at approximately 35° C., with air flow and particle motion for 20 minutes. A further sub-sample (1A) was taken and placed into a traditional dryer and dried overnight (20 to 24 hours) at approximately 35° as per standard technique. Further samples 1C and 1D were also taken and dried at approximately 48° C., with air flow and particle motion for 15 minutes and 20 minutes respectively.

Before drying, the moisture content of each sample was measured as having a moisture content of 32.1% wt.

After the times defined above, the samples were measured for phosphorus levels (Olsen P). Phosphorus tests were chosen as a representative indicator as phosphorus is an important agronomical test for pastoral farming as phosphate fertiliser incurs the majority of the cost of fertilisation. Further, the Olsen P is a relatively sensitive test compared with other mineral and physical characteristic tests and hence is representative of the accuracy that may be attained by the method of the present invention.

The moisture content after drying in the case of sample 1A, the traditional method, was 0.0% wt. For samples 1B, 1C and 1D, the residual moisture contents were 2.2% wt, 0.2% wt and 0.0% wt respectively.

Olsen phosphorus (P) levels after drying were measured in duplicate and shown in Table 1 below.

TABLE 1
Olsen P Levels Example 1

| | Olsen P - | Olsen P - |
|---|---|---|
| Soil Sample | Test 1 | Test 2 |
| 1A | 45 | 45 |
| Traditional 20 to 24 hour drying, 35° C. | | |
| 1B | 45 | 46 |
| 35° C., airflow, motion, 20 minutes | | |
| 1C | 51 | 54 |
| 48° C., airflow, motion, 20 minutes | | |
| 1D | 47 | 50 |
| 48° C., airflow, motion, 15 minutes | | |

### EXAMPLE 2

The same soil type as Example 1 was tested using different samples and the same method as described in Example 1 with soil samples 1 labelled 2A (traditional drying at 35° C. overnight), 2B (35° C., with air flow and particle motion for 20 minutes) and 2C and 2D (48° C., with air flow and particle motion for 20 and 15 minutes respectively).

Before drying, the moisture content of each sample was measured as having a moisture content of 31.1% wt.

The moisture content after drying in the case of sample 2A, the traditional method, was 0.0% wt. For samples 2B, 2C and 2D, the residual moisture content was 3.7% wt, 0.0% wt and 0.2% wt respectively.

Olsen phosphorus (P) levels after drying were measured in duplicate and shown in Table 2 below.

TABLE 2
Olsen P Levels Example 2

| | Olsen P - | Olsen P - |
|---|---|---|
| Soil Sample | Test 1 | Test 2 |
| 2A | 20 | 22 |
| Traditional, 20 to 24 hours, 35° C. | | |
| 2B | 20 | 21 |
| 35° C., airflow, motion, 20 minutes | | |
| 2C | 23 | 22 |
| 48° C., airflow, motion, 20 minutes | | |
| 2D | 23 | 24 |
| 48° C., airflow, motion, 15 minutes | | |

### EXAMPLE 3

The same soil type as Example 1 was tested using different samples and the same method as described in Example 1, with soil samples 1 labelled 3A (traditional drying at 35° C. overnight), 3B (35° C., with air flow and particle motion for 20 minutes), and 3C and 3D (48° C., with air flow and particle motion for 20 and 15 minutes respectively).

Before drying, the moisture content of each sample was measured as having a moisture content of 31.1% wt.

The moisture content after drying in the case of sample 2A, the traditional method, was 0.0% wt. For samples 3B, 3C and 3D, the residual moisture content was 7.4% wt, 0.4% wt and 2.4% wt respectively.

Olsen phosphorus (P) levels after drying were measured in duplicate and shown in Table 3 below.

TABLE 3
Olsen P Levels Example 3

| | Olsen P - | Olsen P - |
|---|---|---|
| Soil Sample | Test 1 | Test 2 |
| 3A | 25 | 25 |
| Traditional, 20 to 24 hours, 35° C. | | |
| 3B | 25 | 28 |
| 35° C., airflow, motion, 20 minutes | | |
| 3C | 33 | 34 |
| 48° C., airflow, motion, 20 minutes | | |
| 3D | 35 | 36 |
| 48° C., airflow, motion, 15 minutes | | |

### EXAMPLE 4

Different soil types were tested, gley soil (silt loam), using the same method as described in Example 1 with soil samples 1 labelled 4A (traditional drying at 35° C. overnight), 4B (35° C., with air flow and particle motion for 20 minutes), and 4C and 4D (48° C., with air flow and particle motion for 20 and 15 minutes respectively).

Before drying, the moisture content of each sample was measured as having a moisture content of 39.5% wt.

The moisture content after drying in the case of sample 4A, the traditional method, was 0.0% wt. For samples 4B, 4C and 4D, the residual moisture content was 8.9% wt, 0.2% wt and 2.8% wt respectively.

Olsen phosphorus (P) levels after were measured in duplicate and shown in Table 4 below.

TABLE 4
Olsen P Levels Example 4

| | Olsen P - | Olsen P - |
|---|---|---|
| Soil Sample | Test 1 | Test 2 |
| 4A | 13 | 13 |
| Traditional, 20 to 24 hours, 35° C. | | |
| 4B | 12 | 12 |
| 35° C., airflow, motion, 20 minutes | | |
| 4C | 14 | 15 |
| 48° C., airflow, motion, 20 minutes | | |
| 4D | 14 | 13 |
| 48° C., airflow, motion, 15 minutes | | |

### EXAMPLE 5

Different soil types were tested, allophanic soil (sandy loam) using the same method as described in Example 1 with soil samples labelled 5A (traditional drying at 35° C. overnight), 5B (35° C., with air flow and particle motion for 20 minutes) and 5C and 5D (48° C., with air flow and particle motion for 20 and 15 minutes respectively).

Before drying, the moisture content of each sample was measured as having a moisture content of 30.9% wt.

The moisture content after drying in the case of sample 5A, the traditional method, was 0.0% wt. For samples 5B, 5C and 5D, the residual moisture content was 5.0% wt, 0.0% wt and 0.4% wt respectively.

Olsen phosphorus (P) levels after drying were measured in duplicate and shown in Table 5 below.

TABLE 5
Olsen P Levels Example 5

| | Olsen P - | Olsen P - |
|---|---|---|
| Soil Sample | Test 1 | Test 2 |
| 5A | 17 | 18 |
| Traditional, 20 to 24 hours, 35° C. | | |
| 5B | 17 | 17 |
| 35° C., airflow, motion, 20 minutes | | |
| 5C | 21 | 21 |
| 48° C., airflow, motion, 20 minutes | | |
| 5D | 21 | 25 |
| 48° C., airflow, motion, 15 minutes | | |

The above examples show that the two methods of preparation compared well with a statistical analysis showing no significant difference in Olsen P levels using either traditional methods of preparation or rapid drying at 35° C. A variation of up to 16% was noted for rapid drying at 48° C. For decisions regarding fertiliser application, this variation is acceptable as only a broad indication is required especially in light of the fact a result can be found with 15 minutes preparation.

The above examples show that the method and device of the present invention allows soil to be dried faster than conventional methods with a useful degree of accuracy in measurement of chemical and physical characteristics, however taking significantly less time for sample preparation than traditional methods.

Aspects of the present invention have been described by way of example only and it should be appreciated that modifications and additions may be made thereto without departing from the scope thereof as defined in the appended claims.

1. A method for drying soil in preparation for analysis comprising the steps of:
(a) increasing the surface area of the soil;
(b) forcing a substantially inert gas through the soil; and
(c) subjecting the soil to an elevated temperature that does not exceed approximately 50° C.

2. The method of claim 1 wherein the sample is prepared for analysis after approximately 1 hour of processing via steps (a) to (c).

3. The method of claim 1 wherein the sample is prepared for analysis after approximately 20 minutes of processing via steps (a) to (c).

4. The method of claim 1 wherein the moisture content after steps (a) to (c) is less than approximately 9% wt.

5. The method of claim 1 wherein said increasing the surface area of the soil as during step (a) comprises breaking the soil down into smaller particles by mechanical motion.

6. The method of claim 1 wherein said increasing the surface area of the soil results in a mean particle size of substantially less than 10 mm.

7. The method of claim 1 wherein said substantially inert gas of step (b) is air.

8. The method of claim 1 wherein said substantially inert gas of step (b) is free of moisture.

9. The method of claim 1 wherein said substantially inert gas of step (b) is conditioned via dehumidification.

10. The method of claim 1 wherein said substantially inert gas of step (b) is conditioned by use of a desiccating agent to remove moisture from the gas.

11. The method of claim 1 wherein the substantially inert gas of step (b) is forced across the soil particles produced from step (a).

12. The method of claim 11 wherein said forcing a substantially inert gas through the soil comprises the use of fan forced substantially inert gas.

13. The method of claim 11 wherein the flow of the forced substantially inert gas is less than 4 m/s.

14. The method of claim 11 wherein the flow of the forced substantially inert gas is approximately 2 m/s.

15. The method of claim 1 wherein the elevated temperature during step (c) is high enough to allow sample drying without impacting on the chemical and/or physical properties to be measured.

16. The method of claim 1 wherein the elevated temperature during step (c) ranges from approximately 20° C. to 50° C.

17. The method of claim 1 wherein the elevated temperature during step (c) ranges from approximately 30° C. to 40° C.

18. The method of claim 1 wherein the elevated temperature during step (c) is approximately 35° C.

19. The method of claim 1 wherein step (c) comprises the use of drying equipment that is preheated.

20. The method of claim 1 further comprising:
(d) moving the soil.

21. The method of claim 20 wherein the particles remain moving for substantially all of the drying time.

22. An assembly for drying of soil comprising:
(a) an inert gas supply device which is capable of forcing inert gas through a soil sample; and
(b) a heating element which is capable of subjecting the soil to an elevated temperature of less than approximately 50° C.

23. The assembly of claim 22 wherein the assembly further includes a soil crusher device which is capable of increasing the surface area of the soil.

24. The assembly of claim 22 where the assembly further includes a device capable of keeping the soil in motion.

25. (canceled)

26. (canceled)

27. The assembly of claim 23 further comprising a device capable of keeping the soil in motion.

28. The method of claim 12 wherein flow of the forced substantially inert gas is approximately 2 m/s.

29. The method of claim 13 wherein the flow of the forced substantially inert gas is approximately 2 m/s.

30. The method of claim 12 wherein the flow of the forced substantially inert gas is less than 4 m/s.
